(19) United States
(12) Patent Application Publication
(10) Pub. No.: US 2013/0011448 A1
(43) Pub. Date: Jan. 10, 2013

(54) SYSTEM FOR IMPROVED DELIVERY OF GENE MODULATING COMPOUNDS

(75) Inventor: Ulf Tedebark, Uppsala (SE)

(73) Assignee: GE HEALTHCARE BIO-SCIENCES AB, Uppsala (SE)

(21) Appl. No.: 13/634,320

(22) PCT Filed: Mar. 15, 2011
(86) PCT No.: PCT/SE2011/050279

§ 371 Date: Sep. 12, 2012

(30) Foreign Application Priority Data
Mar. 16, 2010 (SE) 1000238-4

(51) Int. Cl. C07D 215/46; C12N 5/071; A61K 9/00; A61K 47/22

(52) U.S. Cl. 424/400; 546/163; 514/788; 435/375

(57) ABSTRACT

The present invention relates to a system for intracellular cargo delivery comprising:

D₍ₙ₎BAC
wherein:
Component D is a amine-containing compound;
n is a number of amines between 2-6,
Component B is a di-, tri or tetra ring system, such as quinoline or naphthalene derivatives which may contain several hetero atoms chosen from N, S, O and P;
Component A is an aliphatic linear or branched hydrophobic chain of at least 4 carbon atoms preferably an epoxide, alkyl halide or an acrylamide with 6-30 carbon atoms or a derivative thereof; and
Component C is an optional targeting moiety, such as homing peptide or aptamer, wherein components D, B, A and C may be coupled to each other via spacer(s).

It also relates to the use of the system in diagnosis of diseases, as research tool and as a targeting system, a composition comprising the system and especially a pharmaceutical composition, a material covered with the system and a material having the delivery systems within the material.

### FIELD OF THE INVENTION

The present invention relates to a system or reagent for intracellular delivery of a cargo, preferably a system for intracellular delivery of oligonucleotides.

It also relates to the use of the system in diagnosis of diseases, as research tool and as a targeting system, a composition comprising the system and especially a pharmaceutical composition, a material covered with the system and a material having the delivery systems into the material.

### BACKGROUND OF THE INVENTION

The hydrophobic plasma membrane constitutes an essential barrier for cells in living animals, allowing the constitutive and regulated influx of essential molecules while preventing access to the interior of cells of other macromolecules. Although being pivotal for the maintenance of cells, the inability to cross the plasma membrane is still one of the major obstacles to overcome in order to progress current drug development.

During the past 40 years, several oligonucleotide (ON)-based methods have been developed with the purpose of manipulating gene expression. The basic method involves the use of bacterial plasmids for expression of genes of interest. In addition, to evaluate functional aspects of different genes, this is a highly appealing strategy to utilize in clinical settings, i.e. gene therapy. Gene therapy was originally thought to serve as corrective treatment for inherited genetic diseases. However, over the past 15 years, experimental gene therapy for cancer has become a frequent application although other acquired diseases have also been investigated [1]. 1. Cross, D. & Burmester, J. K. Gene therapy for cancer treatment: past, present and future. Clin Med Res 4, 218-27 (2006).

Other versatile approaches utilizing shorter ON-sequences to interfere with gene expression have emerged. Antisense approaches based on short interfering RNAs (siRNAs) that are utilized to confer gene silencing and splice correcting ONs (SCOs), applied for the manipulation of splicing patterns, have recently been rigorously exploited [2,3]. Although being efficient compounds for regulating gene expression, their anionic hydrophilic nature prohibits cellular internalization. 2. Kim, D. H. & Rossi, J. J. Strategies for silencing human disease using RNA interference. Nat Rev Genet 8, 173-84 (2007).3. Mercatante, D. R., Sazani, P. & Kole, R. Modification of alternative splicing by antisense oligonucleotides as a potential chemotherapy for cancer and other diseases. Curr Cancer Drug Targets 1, 211-30 (2001).

Despite the great potential gene therapy holds for future treatment of various disorders, it suffers from some severe drawbacks. First, plasmids are large, usually exceeding one MDa in size, making them impermeable over cellular membranes. Secondly, viruses have been used to confer cellular internalization of therapeutic genes in clinical trials. Albeit providing an effective means of delivering genes, they might cause severe immunological responses. Thus, in order to progress current gene therapy, safer delivery systems are required, preferably not reliant on the use of viruses.

The search for efficient non-viral delivery vectors has therefore intensified. In the field today, the vectors based on cationic liposomes or polycationic polymers have been employed and these are highly efficient for transfection of commonly used cell lines. However, a great number of these vectors are either sensitive to serum proteins, are unable to transfect the entire cell populations, are inefficient in “hard to transfect” cells, or are simply too toxic. For the vectors on the market today it seems to be a direct correlation between high efficacy and high cytotoxicity. Therefore, there is an urgent need to find delivery vehicles that can overcome the above mentioned problems.

Cell-penetrating peptides (CPPs) are a class of peptides that has gained increasing focus the last years. This ensues as a result of their remarkable ability to convey various, otherwise impermeable, macromolecules across the plasma membrane of cells in a relatively non-toxic fashion, as reviewed in [4]. The peptides are usually less than 30 amino acids (aa) in length with a cationic and/or amphipathic nature and have been extensively applied for delivery of various ONs both in vitro and in vivo [5]. Even though the peptides are non-toxic in general, there are some problems associated with their use [6]. One shortcoming with the CPP technology, in terms of ON-delivery, is that peptides usually need a covalent attachment to ONs, which is a cumbersome procedure and high concentrations of peptide conjugates are generally needed to obtain a significant biological response [7,8]. A few studies have convincingly shown that a non-covalent co-incubation strategy of simply mixing CPPs with ONs works efficiently and in a non-toxic fashion. When using the co-incubation strategy with unmodified CPPs, it seems that the complexes remain trapped inside endosomes and are therefore unable to exert a biological response [9]. Ideally, CPPs would be designed to more efficiently escape endosomal compartments following endocytosis thereby allowing them to be non-covalently complexed with oligonucleotides or plasmids. Attempts have been made to combine the use of CPPs with known transfection reagents to reduce the amount of transfection regent needed to obtain biological responses or CPPs have been co-added with known fusogenic peptides. Another strategy has been to co-add the lysosomotrophic agent chloroquine at high concentrations to increase the efficacy of the CPP/ON complexes, which significantly increases transfections but is limited to in vitro use and furthermore, the high concentrations of chloroquine needed raises toxicity concerns. 4. EL-Andaloussi, S., Holm, T. & Langel, Ü. Cell-penetrating peptides: mechanisms and applications. Curr Pharm Des 11, 3597-611 (2005).5. Mae, M. & Langel, U. Cell-penetrating peptides as vectors for peptide, protein and oligonucleotide delivery. Curr Opin Pharmacol 6, 509-14 (2006).6. El-Andaloussi, S., Jarver, P., Johansson, H. J. & Langel, U. Cargo-dependent cytotoxicity and delivery efficacy of cell-penetrating peptides: a comparative study. Biochem J 407, 285-92 (2007).7. Abes, S. et al. Vectorization of morpholino oligomers by the (R-Ahx-R)4 peptide allows efficient splicing correction in the absence of endosomolytic agents. J Control Release 116, 304-13 (2006).8. Bendifallah, N. et al. Evaluation of cell-penetrating peptides (CPPs) as vehicles for intracellular delivery of antisense peptide nucleic acid (PNA). Bioconjug Chem 17, 750-8 (2006).9. Lundberg, P., El-Andaloussi, S., Sutlu, T., Johansson, H. & Langel, U. Delivery of short interfering RNA using endosomolytic cell-penetrating peptides. Faseb J 21, 2664-71 (2007).

A related patent, US 2007/0059353 discloses a liposome having cellular and nuclear entry ability. The provided liposome has on its surface a peptide comprising multiple consecutive arginine residues, and specifically a liposome is provided wherein the peptide is modified with a hydrophobic group or hydrophobic compound and the hydrophobic group or hydrophobic compound is inserted into a lipid bilayer so that the peptide is exposed on the surface of the bilayer. The problem with this delivery system, apart from the difficulty of constructing such complex vectors, is that they are based on liposomes. Several groups have reported on alterations in gene expression profiles after transfections with liposome-based delivery systems which greatly hamper their use. In addition, oligoarginines are prone to remain bound to endosomal compartments and are therefore not optimal for delivery. An improved strategy would be to chemically modify newly designed or existing CPPs with one or more chemical entities that could promote endosomal escape.

The drug of choice today for endosomal escape is Chloroquine (CQ) and its analogues. It is a, as it is also called, lysosomotropic agent, inhibiting endosome acidification, leading, at higher concentrations, to endosomal swelling and rupture.

There are several U.S. patents disclosing chloroquine for use against a variety of diseases either alone or in combination with other drugs. For instance, U.S. Pat. No. 4,181,725 and A. M. Krieg, et al, U.S. Patent Applic. 20040009949 disclose the use of chloroquine for treating various autoimmune diseases in combination with inhibitory nucleic acids.

The ability of chloroquine to act as “lysosomotropic” agent to enable release of substances from cellular endosomes/lysosomes is well-documented. [Marches, 2004; A. Cuatraro 1990 etc]. Nevertheless, in vivo use of chloroquine was claimed to be prohibited by its toxicity, as high concentration of free chloroquine needs to be administrated to reach endosomes. (Citing J. M. Benns, et al, 1.sup.st paragraph, Bioconj. Chem. 11, 637-645, (2000): “Although chloroquine has proven to aid in the release of the plasmid DNA into the cytoplasm, it has been found to be toxic and thus cannot be used in vivo.”)

Recent US 20070166281 entitled “Chloroquine coupled antibodies and other proteins with methods for their synthesis” discloses coupling of chloroquine and thereof derived structures to different carrier compositions that contain biocleavable linkages allowing release of chloroquines under controlled conditions. US20070166281 is aimed to provide controlled release of the chloroquines from protein or peptide active agent or antibody after the carrier has reached its site of action.

US2006/0040879 Kosak and colleagues discloses compositions and methods for preparing chloroquine-coupled nucleic acid compositions. The prior art has shown that chloroquine given as free drug in high enough concentration, enhances the release of various agents from cellular endosomes into the cytoplasm. The purpose of these compositions is to provide a controlled amount of chloroquine at the same site where the nucleic acid needs to be released, thereby reducing the overall dosage needed. This patent is aimed at achieving controlled release of chloroquine conjugated to nucleic acid compositions, this is not the subject of the present invention, but rather to enhance and simplify delivery in gene therapy in vitro and in vivo.

Two recent publications by Anderson et al (Nat Biotech, vol 26 no 5, page 561-9) “A combinatorial library of lipid-like materials for delivery of RNA therapeutics” and (PNAS 2010 107:1864-1869) “Lipid-like materials for low-dose in vivo gene silencing” describes a combinatorial approach to evaluate lipid-like materials comprising aliphatic chains attached to an amine comprising at least two nitrogens. These lipidoids (>1200 diverse compounds) have then been screened for local delivery of RNAi therapeutics and several exhibited promising properties and were further evaluated for systemic delivery in mice, rats and non-human primates.

Co-pending application PCT/SE2009/051032 relates to improved systems for intracellular delivery of cargo, such as nucleic acids. The systems comprise constructions with modified cell penetrating peptides, As described in said application, because the attachment of four copies of the ring system, component B increases the local effect of the chloroquine analogues. This is a valuable property for in vivo applications. Also, by conjugating chloroquine to the peptide, the effective concentration is reduced by more than a log, most likely explaining the lack of toxicity otherwise seen with chloroquine at 100 μM concentrations. The delivery system makes it possible to release ONs (as cargoes) at the correct intracellular location without addition of extra chloroquine.

### SUMMARY OF THE INVENTION

The present invention provides a system for intracellular cargo delivery comprising a new series of molecules expected to overcome the described drawbacks for non-covalent gene-delivery, ie low and heterogeneous delivery as well as toxicity. The system according to the present invention comprises irreversibly chloroquine analogue coupled compounds including aliphatic chains.

The system comprises chloroquine analogues attached to an amine and further derivatized with aliphatic chains which can presumably be utilized for efficient delivery of a wide variety of ONs, without the toxicity of the delivery agents on the market today. The present invention can presumably both efficiently deliver the drug load into all of the cells in a population as well as releasing the ONs from their entrapment in endosomes. The scope of the invention is described in the appended claims. Preferred embodiments of the invention describe a chloroquine analogue modified with alkylated amines and their applications; enhanced transfection, splice correction as well as siRNA delivery.

### DETAILED DESCRIPTION

The present invention relates to a system designed for intracellular cargo delivery comprising at least one component A chosen from aliphatic linear or branched moieties with at least 4 carbon and a component B chosen from cyclic ring systems comprising 2-4 rings which may contain several hetero atoms chosen from N, S, O and P, wherein component A and B are attached to component D an amine containing compound, and in which said delivery system is capable of delivering a cargo by covalent or non-covalent attachment. The delivery system is called QFect.

In a first aspect, the invention relates to a system for intracellular cargo delivery comprising
D₍ₙ₎BAC
wherein:
Component D is a amine-containing compound;
n is a number of amines of at least 2,
Component B is a di-, tri or tetra ring system, such as quinoline or naphthalene derivatives which may contain several hetero atoms chosen from N, S, O and P;
Component A is an aliphatic linear or branched hydrophobic chain of at least 4 carbon atoms preferably an epoxide or an alkyl halide or an acrylamide with 6-30 carbon atoms or a derivative thereof; and
Component C is an optional targeting moiety,
wherein components D, B, A and C may be coupled to each other via spacer(s).

The targeting moiety C enables the system to reach specific cells or tissue of interest. The targeting moiety may be an aptamer or a targeting peptide such as a homing peptide or a receptor ligand.

According to another embodiment the delivery system further comprises a cargo, which may be delivered into cells, tissue or across a cell layer.

One or more components A, one or more components B, one or more components C and one or more cargos can be coupled covalently to an amino function of component D the amine containing compound. In some delivery systems (QFect's), a branched tree-like structured spacer may be applied. The targeting moiety C may be added non-covalently or through covalent conjugation.

Moreover, one or more of the components A, B, C and the cargo may be attached to one or more components D, an amine containing compounds via a spacer arm. According to the invention, the delivery system may comprise one ore more components A, one or more components B, one or more targeting components C coupled to each other in any order without any cargo. One or more components A may be coupled to one or more components B in any order prior coupling to component D, an amine containing compound and without any targeting components C and without any cargoes. These may be delivered for further coupling of cargoes at a later stage. The invention relates to a method of delivering cargoes into a target cell in vivo or in vitro by using such a delivery system.

The invention also relates to the method how to produce the QFect constructs.

### Component D

Component D can be an amine, preferably with at least two primary nitrogens and zero or at least one secondary amine and zero or at least one tertiary amine and zero or at least one quaternary amine where the primary amines are separated by at least two atoms, linear, cyclic, polycyclic or branched and additional amines may be located anywhere in the compound. Component D may also include one to several other functional groups such as but not limited to —OH, —SH, —NHCOR where R=alkyl, aryl, H, —CONRR′ where R, R′=independently chosen from H, alkyl or aryl.

Thus, component D is an amine containing compound with at least two amines separated by any type of atoms in any order including branched, cyclic or polymeric structures with any additional functional groups. Examples of amine containing compounds:

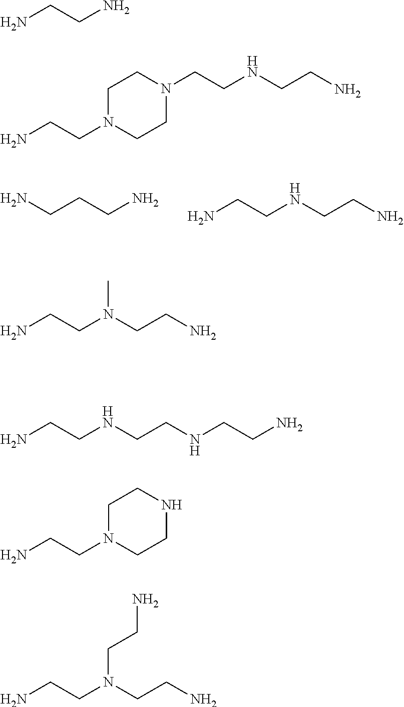

### Component A

Component A can be one or several aliphatic linear or branched moieties with at least 4 carbon.

The aliphatic component A may be 4-30 carbon atoms and preferably contain an epoxide or alkylhalide or acrylamide or most preferably a terminal epoxide. Such an aliphatic epoxide may comprise 4, 5, 6, 7, 8, 9, 10, 11, 12, 13, 14, 15, 16, 17, 18, 19, 20, 21, 22, 23, 24, 25, 26, 27, 28, 29, 30 carbon atoms or any interval created by these figures. It may also be a derivative thereof. Moreover the chain may contain a mix of saturated/non-saturated bonds.

### Component B

The ring systems component B comprising 2-4 rings which may contain several hetero atoms chosen from N, S, O and P, wherein component(s) B is(are) covalently attached to component

D, an amine containing compound. In addition, the component B, may also be one or more copies of a two-four fused cyclic system of 2 to 4 rings of 3- to 8 membered rings, saturated or non-saturated, possibly comprising one to several hetero atoms in the ring systems chosen from N, S, O, B or P. These may be but not limited to biphenyl, diphenyl ether, amine, sulphide or peri-and/or ‘ortho-f used and be chosen from but not limited to quinoline, isoquinoline, quinoxaline, pentalene, naphthalene, heptalene, octalene, norbonane, adamantane, indole, indoline, azulene, benzazepine, anthracene, biphenylene, triphenylene and benzanthracene and analogues thereof. Such analogues may comprise one or more carboxylic groups and/or one or more additional functional groups such as but not limited to one or more amines, one or more thiols, one or more hydroxyls, one or more esters and one or more aldehydes.

According to one embodiment, several copies of component A may be conjugated on a side chain residue via a lysine branched spacer.

These ring systems may also be substituted e. g with other groups with pH buffering capacity to destabilize endosomes or a as a condensing moiety for nucleotide interactions. Examples of substituents but not limited to, could be one to several primary, secondary and/or tertiary amines, substituted or included in as any aliphatic or aromatic moiety or combinations thereof, also spaced by zero to several atoms in a linear, branched or cyclic fashion or a combination thereof.

More preferred examples are N′-(7-chloroquinolin-4-yl)-N,N-diethyl-pentane-1,4-diamine (chloroquine) or derivatives thereof, di- to tetra ring systems (naphthalene and/or biphenyl connected), 4- to 8 membered rings, one to several hetero atoms in ring systems attached anywhere to the construct. Most preferred example is 7-(trifluoromethyl)-quinolin-4-yl. They may have spacer arm(s) of different lengths.

### Component C

The C component is a targeting moiety, such as a ligand for a known or unknown receptor. The substrate may be an aptamer and/or targeting peptide.

An aptamer is a double stranded DNA or single stranded RNA molecule that bind to a specific molecular targets, such as a protein or metabolite.

A targeting peptide is a peptide that binds to specific molecular targets, such as a protein or metabolite, for example a homing peptide. A homing peptide is a peptide squence which have been selected to bind a certain tissue or cell type, ususally by phage display.

In addition, the component C may be another molecule that directs the delivery system to a certain cell type or tissue; well known examples are over-expression of growth factors as tumour targets.

The targeting moiety C may also be non-covalently complexed with the component A and B of the delivery system, as a part of a composition.

Generally, a cell-selective compound will be useful in the targeted transport of any kind of drug or pharmaceutical substance to a variety of specific eukaryotic and/or prokaryotic cellular targets. A cell-selective transport of such cargo is e.g. envisioned for an improved treatment or prevention of infectious diseases, such as diseases caused by a viral, bacterial or parasital infection.

In yet a further embodiment of the present invention, a compound is provided that will enter selectively into a certain cell type/tissue/organ, or that transports a cargo that will only be activated in a certain cell type, tissue, or organ type.

### Spacer

Spacers may be used for the attachment of component A, B and C and the cargo to component D, the amine containing compound.

According to one embodiment the spacer comprises one or more amino acids e.g. lysine units, such as 1, 2, 3, 4, 5, 6, 7, 8, 9, 10 amino acids. e.g. lysine units, which may be straight or branched and modified with functional groups or extra carbon atoms for further attachment.

The spacer may be a linear or branched moiety with one to several substituents facilitating attachment of component B or a tree-like structure comprised of preferably but not limited to lysine or ornithine residues arranged in a dendrimeric fashion comprising of 1, 2, 3, 4 or unlimited number of Lys or Orn residues. The Tree may have any number of branches and is comprised of any number of branching units such as lysine or ornithine residues; “Nic” can be nicotinic acid, benzoic acid, quinolinic acid, naphthalene carboxylic acid, chloroquine or its derivative, or any other organic molecule.

A spacer is preferable used in conjugating two-four copies of the ring system (component B) to component D, the amine containing compound. The spacer may be a dendrimer.

Dendrimers are repeatedly branched molecules, in this case preferable with a peptide backbone.

Moreover, of great importance, only very low amounts of delivery agent and ONs are needed to gain a biological response, which decrease both labours and costs.

The QFect analogues may be further chemically modified. Instead of, or in addition to, being modified with component A, these may also be conjugated to a lysine tree bearing e.g. one or more such as four B components that facilitates release of the ONs from vesicular compartments. These are not only active for transportation of ON compounds acting in the nucleus of cells but can additionally be efficiently utilized for the delivery of cytoplasmically active ONs such as anti-miRs and siRNAs.

Preferred QFect systems comprises the following structures including analogues from mono-alkylated to fully alkylated derivatives (not depicted), where Cn equals an interval of carbon atoms from 6 to 28 which may be branched, linear or a mix thereof included non-saturated carbon and other functional groups. Also included are structures where epoxide opening was on the beta carbon (not depicted).

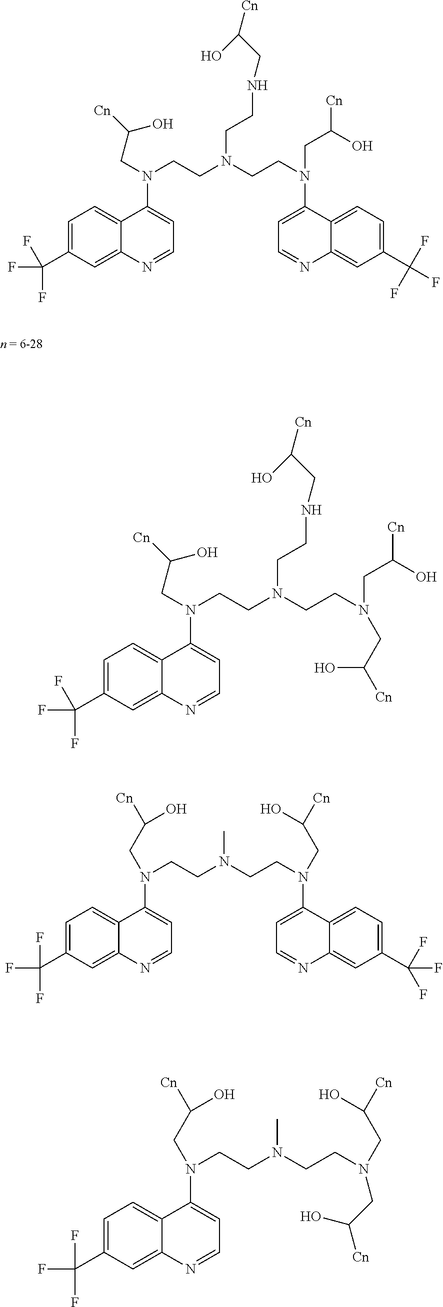

### The Cargo

The cargo may be chosen from gene modulating compounds, such as oligonucleotides or plasmids. They may be attached to the delivery system by covalent attachment or complex formation.

The family of oligonucleotides includes antisense oligonucleotides for mRNA silencing, splice correcting oligonucleotides for manipulation of pre-mRNA splicing patterns, and short interfering RNAs for gene silencing.

The cargo may be selected from the group consisting of oligonucleotides and modified versions thereof, single strand oligonucleotides (DNA, RNA, PNA, LNA and all synthetic oligonucleotides), double-strand oligonucleotides (siRNA, shRNA, decoy dsDNA etc.), plasmids and other varieties thereof, synthetic nucleotide analogs for the purpose of inhibition of viral replication or antiviral ONs.

The cargo may also comprise a cell- or tumour homing peptide, aptamer, a receptor ligand, a spacer comprising a cleavable site coupled to an inactivating peptide, peptide ligands, cytotoxic peptides, bioactive peptides, diagnostic agents, proteins, pharmaceuticals e.g.anticancer drugs and antibiotics, circulation clearance modifiers, like PEG.

It has been estimated that 20-30% of all disease-causing mutations affects pre-mRNA splicing. Several genetic disorders and other diseases, including β-thalassemia, cystic fibrosis, muscular dystrophies, cancers, and several neurological disorders, are associated with alterations in alternative splicing, reviewed in. The majority of mutations that disrupt splicing is single nucleotide substitutions within the intronic or exonic segments of the classical splice sites. These mutations result in either exon skipping, use of a nearby pseudo 3′- or 5′splice site, or retention of the mutated intron. Mutations can also introduce new splice sites within an exon or intron.

One of the first splicing mutations described was found in β-thalassemia patients, where mutations in intron 2 of β-globin pre-mRNA create an aberrant 5′ splice site, concomitantly activating a cryptic 3′ splice site. This in turn leads to an intron inclusion and non-functional protein. Same type of mutations has been identified in the cystic fibrosis transmembrane conductance regulatorgene, resulting in aberrant splicing and development of cystic fibrosis. Duchenne muscular dystrophy (DMD), characterized by progressive degenerative myopathy, and its milder allelic disorder, Becker muscular dystrophy (BMD), are both caused by mutations in the dystrophin gene. Most nonsense mutations within this gene result in premature termination of protein synthesis and to the severe DMD, whereas a nonsense mutation within a regulatory sequence generates partial in-frame skipping of an exon and is associated with the milder BMD. Also, several types of cancers are known to emenate from mutations affecting alternative splicing. Thus, by using oligonucleotides that sequence specifically binds to these intronic/exonic mutations, these mutations are masked and splicing restored.

Further, the invention relates to a method of delivering cargoes into a target cell in vivo or in vitro. Formation of the complex between QFect and the ONs and optionally some additives (to facilitate complex formation) described here (siRNA, plasmid, SCO (splice correcting Ons)) may be carried out in a small volume of sterile water 30 minutes in RT, and then added, in most experiments, in full serum containing media.

The cargo may also be selected from a fluorescent marker, a cell- or a linker comprising a cleavable site coupled to an inactivating peptide, peptide ligands, cytotoxic peptides, bioactive peptides, diagnostic agents, proteins, pharmaceuticals e.g.anticancer drugs, antibiotica, chemotherapeutics.

The cargo may be attached to any of the components A, B and/or C by covalent or non-covalent bonds. According to one embodiment the cargo may be attached to component D, the amine containing compound. In one embodiment of the invention, the compound may be coupled by a S-S bridge to said cargo. Naturally, there are a broad variety of methods for coupling a cargo to the compound, selected individually depending on the nature of compound, cargo and intended use. A mode for coupling can be selected from the group consisting of covalent and non-covalent binding, as biotin-avidin binding, ester linkage, amide bond, antibody bindings, etc.

The anticancer drugs may be an alkylating agent, an antimetabolite and a cytotoxic antibiotic.

The alkylating agent may include 4[4-Bis(2-chloroethyl)amino)phenyl]butyric acid (chlorambucil) or 3-[4-(Bis(2-chloroethyl)amino)phenyl]-L-alanine (Melphalan), the antimetabolite is N-[4-(N-(2,4-Diamino-6-pteridinylmethyl)methylamino)-benzoyl]-L-glutamic acid (Methotrexate) and the cytotoxic antibiotic is (8S,10S)-10-[(3-Amino-2,3,6-trideoxy-a-L-lyxo-hexopyranosyl)oxy]-8-glycoloyl-7,8,9,10-tetrahydro-6,8,11-trihydroxy-1-methoxy-5,12-naphthacenedione (Doxorubicin).

The delivery system may further comprise at least one imaging agent and/or labelling molecule and/or chemotherapeutics. The delivery system of the invention may then be used as chemotherapeutics and/or imaging agents. Such composition may possibly also comprise targeting sequences. The chemotherapeutics and/or imaging agent may be used for delivery of antiviral oligonucleotides.

The labelling molecules may be molecular beacons, including quenched fluorescence based beacons and FRET technology based beacons, for labelling or quantification of intracellular mRNA.

Molecular beacons are molecules, e.g. single-stranded oligonucleotides, with internal fluorophore and a corresponding quenching moiety organized in a hair-pin structure so that the two moieties are in close proximity. Upon binding a target nucleic acid sequence or exposure to other structural modification, the fluorophore is set apart from the quenching moiety resulting in possibility to detect the fluorophore. The most commonly used molecular beacons are oligonucleotide hybridisation probes used for detection of specific DNA or RNA motifs. Similarly, FRET probes are a pair of fluorescent probes placed in close proximity. Fluorophores are so chosen that the emission spectrum of one overlaps significantly with the excitation spectrum of the other. The energy transferred from the donor fluorophore to the acceptor fluorophore is distance-dependent and therefore FRET-technology based beacons can be used for investigating a variety of biological phenomena that produce changes in molecular proximity of the two fluorophores.

The delivery system may also be conjugated to, or complexed with circulation clearance modifiers, like PEG. Such systems may be used for retarded delivery of cargoes. Circular clearance modifiers are molecules that prolong the half-life of drugs in the body, examples are pegyl, albumin binding or sequence capping.

The delivery system may be used in diagnosis of diseases, as research tool and as a targeting system.

The invention also relates to a composition comprising one or more delivery system as defined herein. In such a composition the delivery systems may comprise different components A, and different components B, and different components D, and/or different targeting components C and/or different cargoes. These delivery systems may comprise different combinations of A, B, C, D and cargo as mentioned above.

The invention also relates to a pharmaceutical composition comprising the delivery system according and/or a composition as defined above.

It also relates to the use of one or more delivery systems for the production of a pharmaceutical composition.

Especially the composition may comprise at least two different delivery systems that may act additative or synergistic. These may be present in the composition in different ratios. For example, the compositions may comprise any combination of QFect based on different component D, amine containing compounds.

Such a pharmaceutical composition may be in the form of a oral dosage unit; an injectable fluid; a suppository; a gel; and a cream and may comprise excipients, lubricants, binders, disintegrating agents, solubilizers, suspending agents, isotonizing agents, buffers, soothing agents, preservatives, antioxidants, colorants, sweeteners.

The invention also relates to a material covered with one or more of the delivery systems according to the invention.

Further, it relates to a biocompatible material having one or more of the delivery systems as described above incorporated into the material. The delivery system according to the invention may be incorporated into the dendrimers, liposomes etc. Liposomes are composite structures made of phospholipids and may contain small amounts of other molecules

As described previously, QFect can be coupled to a cargo to function as a carrier of said cargo into cells, various cellular compartments, tissue or organs. The cargo may be selected from the group consisting of any pharmacologically interesting substance, such as a peptide, polypeptide, protein, small molecular substance, drug, mononucleotide, oligonucleotide, polynucleotide, antisense molecule, double stranded as well as single stranded DNA, RNA and/or any artificial or partly artificial nucleic acid, e.g. PNA, a low molecular weight molecule, saccharid, plasmid, antibiotic substance, cytotoxic and/or antiviral agent. Furthermore, the transport of cargo can be useful as a research tool for delivering e.g. tags and markers as well as for changing membrane potentials and/or properties, the cargo may e.g. be a marker molecule, such as biotin.

With respect to the intended transport of a cargo across the blood-brain barrier, both intracellular and extracellular substances are equally preferred cargo.

All specific details relate mutatis mutandis to all embodiments described herein. When for example specific chemical components are described in relation to the components A, B, D, and C of the delivery system, these also applies when the delivery system is incorporated into the dendrimers or liposomes, a material covered with the delivery system as well as when a delivery system it is covered with circulation clearance modifiers.

### Examples & Experiments

### Example 1

### Synthesis amino-chloroquine derivative, N-(2-aminoethyl)-N-methyl-N′-[7-(trifluoromethyl)-quinolin-4-yl]ethane-1,2-diamine.

A mixture of B, 2.5 g (16.3 mmol) 4-chloro-7-(trifluoromethyl)quinoline and 6 times molar excess of D, N-methyl-2,2′-diaminodiethylamine (8.3 ml) in a 50 mL round-bottom flask equipped with a magnetic stirrer is heated using PEG 400 bath from room temperature to 80° C. over 2.5 h with stirring, then temperature is raised to 130² C over the period of 3 h, and finally heated 2.5 h at 130° C. The reaction mixture is cooled down to room temperature, and cold DCM is added, causing immediate precipitation, which is filtered off. The organic layer is washed twice with 5% aqueous NaHCO₃, then washed twice by water. The organic phase is dried over anhydrous Na₂SO₄, and solvent is removed under reduced pressure (rotavapor). LC-MS analysis of the crude product revealed two major peaks (m/z 313.4 and 508.6) corresponding to monosubstituted (MW 312.3) and the disubstituted amine (MW 507.5) which is used without further purification for derivatization with component A, an epoxide containing aliphatic compound.

### Example 2

### Synthesis amino-chloroquine derivative, Bis N-(2-aminoethyl)-N′-[7-(trifluoromethyl)-quinolin-4-yl]ethane-1,2-diamine.

A mixture of B, 2.5 g (10.8 mmol) 4-chloro-7-(trifluoromethyl)quinoline and 6 times molar excess of D, 2-2′,2″-triaminodiethylamine (9.7 ml) in a 50 mL round-bottom flask equipped with a magnetic stirrer is heated using PEG 400 bath from room temperature to 80° C. over 2.5 h with stirring, then temperature is raised to 130° C. over the period of 3 h, and finally heated 2.5 h at 130° C. The reaction mixture is cooled down to room temperature, and cold DCM is added, causing immediate precipitation, which is filtered off. The organic layer is washed twice with 5% aqueous NaHCO₃, then washed twice by water. The organic phase is dried over anhydrous Na₂SO₄, and solvent is removed under reduced pressure (rotavapor). LC-MS analysis of the crude product revealed two major peaks (m/z 342.4 and 537) corresponding to monosubstituted MW 341.3) and the disubstituted amine (MW 536.5) which is used without further purification for derivatization with component A, an epoxide containing aliphatic compound.

Derivatization with an Alkyl Epoxide

The quinoline (B) derivatized amine (D) from example 1 was reacted with an alkyl epoxide (A). The excess amount of (A) was calculated as the number of primary amines×2 plus the number of secondary amines minus the number of B and optionally minus 1. The BD compound was mixed with A in a sealed tube or flask and heated at 80-100° C. for 3 days.

### Example 3

Part of the crude product (300 mg) from example 1 above was treated with either 1.2-epoxytetradecane (85%, 1.03 mL, 3.5 mmol) or 1.2-epoxyhexadecane (85%, 1.16 mL, 3.5 mmol) at 90 C for 3 days. LC-MS analysis of the crude reaction mixture revealed peaks corresponding to monoquinolated tri-, di- and monoepoxyalkylated products and, peaks corresponding to diquinolated di- and monoepoxyalkylated products.

### Example 4

Part of the crude product (300 mg) from example 2 above was treated with either 1.2-epoxytetradecane (85%, 1.54 mL, 5.2 mmol) or 1.2-epoxyhexadecane (85%, 1.74 mL, 5.2 mmol) at 90 C for 3 days. LC-MS analysis of the crude reaction mixture revealed peaks corresponding to monoquinolated tetra-, tri-, di- and monoepoxyalkylated products and, peaks corresponding to diquinolated tri-, di- and monoepoxyalkylated products.

### REFERENCES

1. A system for intracellular cargo delivery comprising
D₍ₙ₎BAC
wherein:
Component D is a amine-containing compound;
n is a number of amines of at least 2,
Component B is a di-, tri or tetra ring system, such as quinoline or naphthalene derivatives which may contain several hetero atoms chosen from N, S, O and P;
Component A is an aliphatic linear or branched hydrophobic chain of at least 4 carbon atoms preferably an epoxide, alkyl halide or an acrylamide with 6-30 carbon atoms or a derivative thereof; and
Component C is an optional targeting moiety, such as homing peptide or aptamer, wherein components D, B, A and C may be coupled to each other via spacer(s).

2. The system of claim 1, wherein Component D comprises an amine containing-compound where n is between 2-6; and Component A comprises a terminal epoxide with 6-30 carbon atoms.

3. The system of claim 1, wherein the number of Component A is no more than two times the number of amines (provided no branched spacer is used).

4. The system of claim 1, wherein Component B is naphtyl or 7-(trifluoromethyl)-quinolin-4-yl or a derivative thereof.

5. The system of claim 1, wherein at least two amines in Component D are secondary and/or primary according to the following formulas:

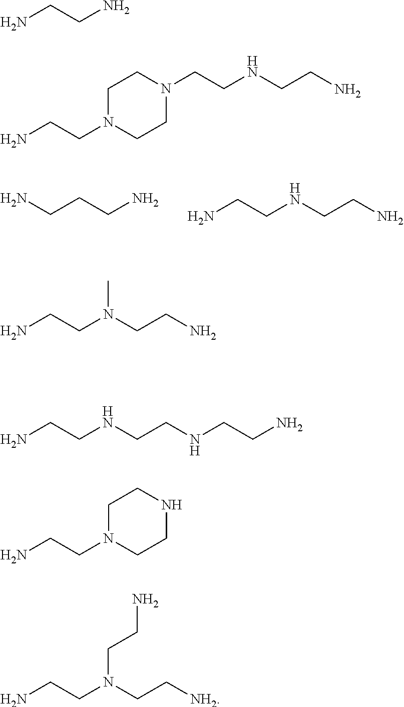

6. The system of claim 1, wherein at least one of the Components A, B, and C are attached to D with a linear or branched spacer arm, 1-8 copies of A or B per spacer.

7. The system of claim 1, comprising the following structures including analogues from mono-alkylated to fully alkylated derivatives (not depicted), where Cn equals an interval of carbon atoms from 6 to 28:

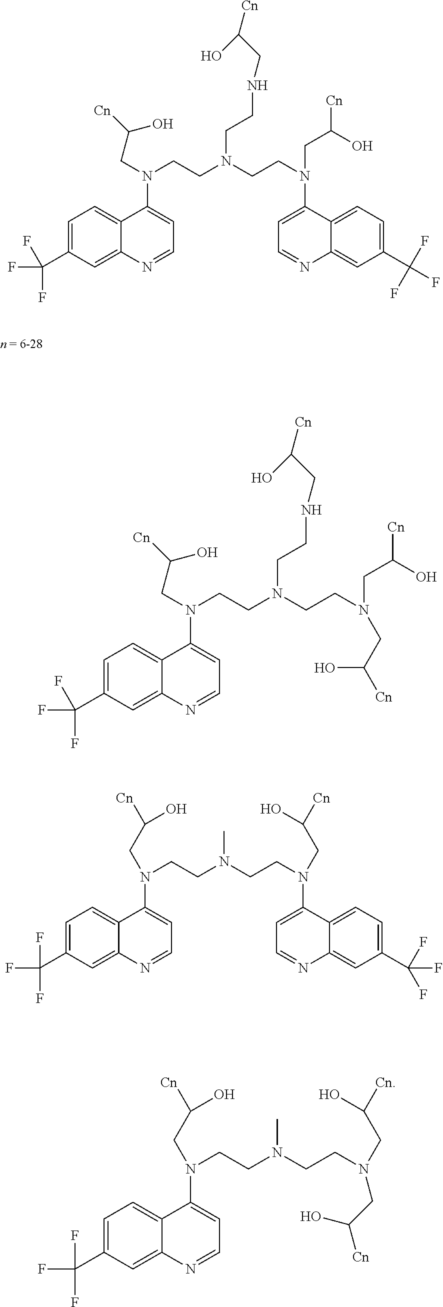

8. The system of claim 7, wherein the cargo is selected from the group consisting of oligonucleotides and modified versions thereof, including single strand oligonucleotides (DNA, RNA, PNA, LNA and all synthetic oligonucleotides), double-strand oligonucleotides (siRNA, shRNA, decoy dsDNA etc.), plasmids and other varieties thereof synthetic nucleotide analogues.

9. The system of claim 7, further comprising at least one imaging agent and/or labelling molecule, such as fluorescent marker.

10. The system of claim 9, wherein the labelling molecules are molecular beacons, including quenched fluorescence based beacons and FRET technology based beacons, for labelling or quantification of intracellular mRNA.

11. (canceled)

12. A composition comprising more than one delivery system of claim 1, wherein the delivery systems comprise different components A, and/or different components B, and/or different components C and/or different cargos and possibly also circulation clearance modifiers, like PEG.

13. A pharmaceutical composition comprising the delivery system of claim 12.

14. A biocompatible material covered with one or more of the delivery systems of claim 1.

15. A biocompatible material having one or more of the delivery systems of claim 1 incorporated into the material.
